(19) United States
(12) Patent Application Publication
(10) Pub. No.: US 2007/0191462 A1
(43) Pub. Date: Aug. 16, 2007

(54) Combination of A 5-HT(1) Receptor Agonist and an Alpha-2-Delta Ligand for the Treatment of Migraine

(75) Inventors: Jayasena Hettiarachchi, (US); Iradi Haji Mohamad Reza, (US); Carolyn Rose Sikes, (US)

Correspondence Address: PHARMACIA CORPORATION;GLOBAL PATENT DEPARTMENT, POST OFFICE BOX 1027, ST. LOUIS, MO, 63006, US

(73) Assignee: Pfizer Limited, Kent (GB)

(21) Appl. No.: 11/682,040

(22) Filed: Mar. 5, 2007

(30) Foreign Application Priority Data
Sep. 7, 2004 (GB) 0419849.5

(51) Int. Cl. A61K 31/4035

(52) U.S. Cl. 514/416

(57) ABSTRACT

The present invention relates to a combination of a 5-HT_{1B}, 5-HT_{1D} or 5-HT_{1F} agonist and an alpha-2-delta ligand. Such a combination is useful in the treatment of pain, particularly the pain arising from migraine.

The present invention relates to a combination of a 5-HT_{1B}, 5-HT_{1D} or 5-HT_{1F} receptor agonist and an alpha-2-delta ligand, as well as to pharmaceutical compositions comprising such a combination and to the uses of such a combination in the treatment of pain and other conditions, especially in the treatment of migraine.

Serotonin (5-hydroxytryptamine, 5-HT) acts at a number of membrane-bound receptors known as 5-HT receptors. These heterogeneous receptors belong to the G-protein coupled receptor superfamily and have been divided into six broad classes (5-HT₁, 5-HT₂, 5-HT₄, 5-HT₅, 5-HT₆ and 5-HT₇). Some of these classes can be further subdivided. The 5-HT, class, for example, comprises five receptor subtypes, all of which have a nanomolar affinity for serotonin. The 5-HT_{1A}, 5-HT_{1B} and 5-HT_{1D} subtypes are characterized by a high affinity for 5-carboxamidotryptamine whilst the 5-HT_{1E} and 5-HT_{1F} subtypes are characterized by a low affinity for this synthetic agonist. See Lanfumey and Hamon in Current Drug Targets—CNS & Neurological Disorders, 2004, 3(1), 1-10 for further information.

A number of indole 5-HT, agonists (commonly known as triptans) have been identified which act most potently at the 5-HT_{1B} and 5-HT_{1D} receptor subtypes and have efficacy in the treatment of migraine. These include sumatriptan, naratriptan, zolmitriptan, rizatriptan, frovotriptan, almotriptan and eletriptan. Ergotamine and dihydroergotamine are also potent agonists of 5-HT_{1B} and 5-HT_{1D} receptors. More recently, selective agonists of the 5-HT_{1F} receptor (such as LY334370 and LY344864) have been discovered and shown to be effective in preclinical models of migraine (see Phebus et al, Society for Neurosceince, 1996, 22, 1331 and Life Sci., 1997, 61, 2117).

An alpha-2-delta ligand (also known as a GABA analogue) is a compound which selectively displaces ³H-gabapentin from brain membranes (e.g. porcine or human brain membranes) and consequently has a high affinity interaction with the alpha-2-delta (α₂δ) subunit of voltage-gated calcium channels. Alpha-2-delta ligands act on voltage-gated calcium channels to attenuate excessive neuronal activity by reducing the depolarization-induced movement of calcium ions into presynaptic terminals and reducing the subsequent release of neurotransmitters such as glutamate, noradrenalin and substance P.

Alpha-2-delta ligands have utility in the treatment of a number of conditions. The best known alpha-2-delta ligand, gabapentin (NEURONTIN®, 1-(aminomethyl)-cyclohexylacetic acid) was first described in the patent family comprising U.S. Pat. No. 4,024,175. The compound is approved for the treatment of epilepsy and neuropathic pain. Although recent clinical trials have shown that gabapentin is efficacious in migraine prophylaxis, there are no reports showing efficacy in the acute (abortive) treatment of migraine.

A second alpha-2-delta ligand, pregabalin (LYRICA®, (S)-(+)-4-amino-3-(2-methylpropyl)butanoic acid), is described in EP-A-0641330 as an anti-convulsant useful in the treatment of epilepsy. The use of pregabalin in the treatment of pain is described in EP-A-0934061. Pregabalin readily crosses the blood-brain barrier through the L-amino acid transporter of cell membranes, thereby reaching its key targets in the brain and spinal cord.

There is an ongoing need to provide better treatments for pain (e.g. migraine headaches) that are, for example, more effective at lower doses, effective against a wider spectrum of pain conditions, less prone to produce side effects, faster acting and longer acting. A lower rate of recurrence in certain painful conditions (e.g. migraine) is also desirable.

The use of a 5-HT_{1B}, 5-HT_{1D} or 5-HT_{1F} receptor agonist (particularly a triptan) in the treatment of migraine is somewhat limited by the need for early administration in order to achieve optimal pain relief and by the potential for unwanted side-effects at therapeutic doses. Migraine is a primary brain disorder in which neural events result in both dilation and inflammation of cranial blood vessels and neurogenic inflammation in the brain. An increased sensitivity and excitability is produced resulting in peripheral sensitization followed by central sensitization. Central sensitization is an increase in the excitability of neurons within the central nervous system, so that inputs that would normally evoke a mild or absent sensation now produce an exaggerated response (e.g. tactile allodynia in which a pain response is evoked by light brushing of the skin). Recent evidence indicates that triptans are more effective if given early in an attack, before peripheral neurons sensitize central neurons leading to central sensitization and that they are unable to reverse ongoing peripheral or central sensitisation.

It has now been surprisingly found that combination therapy with a 5-HT_{1B}, 5-HT_{1D} or 5-HT_{1F} receptor agonist and an alpha-2-delta ligand offers significant benefits in the treatment of pain, particularly in the treatment of migraine. Such combination therapy is particularly advantageous when compared with therapy using either agent alone. Such a combination of a 5-HT_{1B}, 5-HT_{1D} or 5-HT_{1F} receptor agonist and an alpha-2-delta ligand results unexpectedly in a synergistic effect, resulting in greater efficacy than would be obtained using either class of agent singly. In particular, the dose of a 5-HT_{1B}, 5-HT_{1D} or 5-HT_{1F} receptor agonist (particularly a triptan) necessary to treat a migraine attack is reduced, potentially leading to fewer side-effects. Furthermore, the efficacy of such a compound, when administered in the later phases of an attack, at a time when peripheral sensitisation has already started, is considerably greater when administered in combination with an alpha-2-delta ligand.

The invention therefore provides a combination of a 5-HT_{1B}, 5-HT_{1D} or 5-HT_{1F} receptor agonist and an alpha-2-delta ligand.

Further, the invention provides a pharmaceutical composition comprising a 5-HT_{1B}, 5-HT_{1D} or 5-HT_{1F} receptor agonist, an alpha-2-delta ligand and a pharmaceutically acceptable excipient, diluent or carrier.

Further, the invention provides a combination of a 5-HT_{1B}, 5-HT_{1D} or 5-HT_{1F} receptor agonist and an alpha-2-delta ligand for use as a medicament.

Further, the invention provides the use of a 5-HT_{1B}, 5-HT_{1D} or 5-HT_{1F} receptor agonist or an alpha-2-delta ligand in the manufacture of a medicament for simultaneous, sequential or separate administration of both agents in the treatment of pain (especially migraine).

Further, the invention provides a combination of a 5-HT_{1B}, 5-HT_{1D} or 5-HT_{1F} receptor agonist and an alpha-2-delta ligand for simultaneous, sequential or separate administration in the treatment of pain (especially migraine).

Further, the invention provides a method of treating pain (especially migraine) comprising administering simultaneously, sequentially or separately, to a mammal in need of such treatment, an effective amount of a 5-HT_{1B}, 5-HT_{1D} or 5-HT_{1F} receptor agonist and an alpha-2-delta ligand.

Further, the invention provides a kit comprising a 5-HT_{1B}, 5-HT_{1D} or 5-HT_{1F} receptor agonist, an alpha-2-delta ligand and means for containing said compounds.

Further, the invention provides a product containing a 5-HT_{1B}, 5-HT_{1D} or 5-HT_{1F} receptor agonist and an alpha-2-delta ligand as a combined preparation for simultaneous, separate or sequential use in the treatment of pain (especially migraine).

The combination provided by the present invention is useful in the treatment of pain, which is a preferred use. Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288,1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating. Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353,1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;

heart and vascular pain, including pain caused by angina, myocardical infarction, mitral stenosis, pericarditis, Raynaud's phenomenon, scleredoma and skeletal muscle ischemia;

head pain, such as migraine (including migraine with aura and migraine without aura), cluster headache, tension-type headache mixed headache and headache associated with vascular disorders; and

orofacial pain, including dental pain, otic pain, burning mouth syndrome and temporomandibular myofascial pain.

The combination of the present invention is potentially useful in the treatment of all kinds of pain, particularly head pain, most particularly migraine, tension type headaches and cluster headaches. All kinds of migraine may be treated, including early migraine, menstrual migraine, migraine in children, mild migraine and recurrent migraine. The combination is useful both in the treatment of migraine and the prevention of migraine recurrence.

The combination of the present invention is also useful in the treatment of conditions other than pain. In particular, the combination provided by the present invention may be useful in the treatment of overactive bladder, premature ejaculation, chronic paroxysmal hemicrania, depression, drug abuse, emesis, eating disorders, hypertension, post-traumatic head and neck injury and obesity and as a vasodilator or antithrombotic agent.

The combination of the present invention may also be useful in the treatment of epilepsy, faintness attacks, hypokinesia, cranial disorders, neuropathalogical disorders and neurodegenerative disorders. Such neurodegenerative disorders include, for example, Alzheimer's disease, Huntington's disease, Parkinson's disease, Amyotrophic Lateral Sclerosis and acute brain injury. Neurodegenerative disorders associated with acute brain injury include stroke, head trauma, and asphyxia. Stroke, which refers to a cerebral vascular disease and is also known as a cerebral vascular accident (CVA), includes acute thromboembolic stroke and both focal and global ischemia. Also included are transient cerebral ischemic attacks and other cerebral vascular problems accompanied by cerebral ischemia.

These vascular disorders may occur in a patient undergoing carotid endarterectomy specifically or other cerebrovascular or vascular surgical procedures in general, or diagnostic vascular procedures including cerebral angiography and the like. Other related incidents are head trauma, spinal cord trauma, or injury from general anoxia, hypoxia, hypoglycemia, hypotension as well as similar injuries seen during procedures from embole, hyperfusion and hypoxia. The present invention would be useful in the treatment of a range of incidents, for example, during cardiac bypass surgery, in incidents of intracranial hemorrhage, in perinatal asphyxia, in cardiac arrest and in status epilepticus.

The combination of the present invention may also be useful in the treatment of depression (e.g. single episodic or recurrent major depressive disorders, dysthymic disorders, depressive neurosis and neurotic depression, melancholic depression including anorexia, weight loss, insomnia, early morning waking or psychomotor retardation, atypical depression or reactive depression, including increased appetite, hypersomnia, psychomotor agitation or irritability, seasonal affective disorder, minor depression and pediatric depression), bipolar disorders or manic depression (e.g. bipolar I disorder, bipolar II disorder and cyclothymic disorder) conduct disorder; disruptive behavior disorder, behavioral disturbances associated with mental retardation, autistic disorder, conduct disorder; anxiety disorders (such as panic disorder with or without agoraphobia, agoraphobia without history of panic disorder, specific phobias such as specific animal phobias, social anxiety, social phobia including social anxiety disorder, obsessive-compulsive disorder and related spectrum disorders and generalised anxiety disorders), stress disorders (including post-traumatic stress disorder, acute stress disorder and chronic stress disorder), borderline personality disorder, schizophrenia and other psychotic disorders, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders (such as severe major depressive disorder), mood disorders associated with psychotic disorders (such as acute mania and depression associated with bipolar disorder), mood disorders associated with schizophrenia, delirium, dementia, senile dementia, memory disorders, loss of executive function, vascular dementia, movement disorders (such as akinesias, dyskinesias, including familial paroxysmal dyskinesias, spasticities, Scott syndrome, PALSYS and akinetic-rigid syndrome), extra- pyramidal movement disorders (such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication- induced postural tremour), addictive disorders and withdrawal syndrome, chemical dependencies and addictions (e.g., dependencies on, or addictions to, alcohol, heroin, cocaine, benzodiazepines, sychoactive substances, nicotine, or phenobarbitol), behavioural addictions (such as an addiction to gambling), ocular disorders (such as glaucoma and ischemic retinopathy), withdrawal syndrome, adjustment disorders (including depressed mood, anxiety, mixed anxiety and depressed mood, disturbance of conduct, and mixed disturbance of conduct and mood), age-associated learning and mental disorders, anorexia nervosa, apathy, attention-deficit (or other cognitive) disorders due to general medical conditions (including attention-deficit disorder (ADD) and attention-deficit hyperactivity disorder (ADHD) and it's recognized sub-types), bulimia nervosa, chronic fatigue syndrome, somatoform disorders (including somatization disorder, conversion disorder, pain disorder, hypochondriasis, body dysmorphic disorder, undifferentiated somatoform disorder and somatoform NOS), incontinence (e.g. stress incontinence, genuine stress incontinence and mixed incontinence), urinary disorders, premature ejaculation, inhalation disorders, obesity (e.g. reducing the weight of obese or overweight patients), oppositional defiant disorder, premenstrual dysphoric disorder (e.g. premenstrual syndrome and late luteal phase dysphoric disorder), sleep disorders (such as narcolepsy, insomnia and enuresis), specific developmental disorders, selective serotonin reuptake inhibition (SSRI) “poop out” syndrome (wherein a patient fails to maintain a satisfactory response to SSRI therapy after an initial period of satisfactory response) and TIC disorders (e.g. Tourette's Disease).

The alpha-2-delta ligand selected for use in the present invention is preferably potent (having a binding affinity of less than 100 nM, preferably less than 10 nM) and selective. In context of the present invention, a selective apha-2-delta ligand is a compound that binds to the gabapentin binding site of the alpha-2-delta (α₂δ) subunit of voltage-gated calcium channels more potently than it binds to any other physiologically important receptor. Such selectivity is preferably at least 2 fold, more preferably at least 10 fold, most preferably at least 100 fold.

Examples of alpha-2-delta ligands suitable for use with the present invention are those compounds generally or specifically disclosed in U.S. Pat. No. 4,024,175 (particularly gabapentin), EP-A-641330 (particularly pregabalin), U.S. Pat. No. 5,563,175, WO-A-97/33858, WO-A-97/33859, WO-A-99/31057, WO-A-99/31074, WO-A-97/291 01, WO-A-02/085839 (particularly (1 R,5R,6S)-6-(Aminomethyl)bicyclo[[3.2.0]hept-6-yl]acetic acid), WO-A-99/31075 (particularly 3-(1-Aminomethyl-cyclohexylmethyl)-4H-[1,2,4]oxadiazol-5-one and C-[1-(1H-Tetrazol-5-ylmethyl)-cycloheptyl]-methylamine), WO-A-99/21824 (particularly (3S,4S)-(1-Aminomethyl-3,4-dimethyl-cyclopentyl)-acetic acid), WO-A-01/90052, WO-A-01/28978 (particularly (1α,3α,5α)(3-amino-methyl-bicyclo[3.2.0]hept-3-yl)-acetic acid), EP-A-0641330, WO-A-98/17627, WO-A-00/76958 (particularly (3S,5R)-3-aminomethyl-5-methyl-octanoic acid), WO-A-03/082807 (particularly (3S,5R)-3-amino-5-methyl-heptanoic acid, (3S,5R)-3-amino-5-methyl-nonanoic acid and (3S,5R)-3-Amino-5-methyl-octanoic acid), EP-A-1178034, EP-A-1201240, WO-A-99/31074, WO-A-03/000642, WO-A-02/22568, WO-A-02/30871, WO-A-02/30881, WO-A-02/100392, WO-A-02/100347, WO-A-02/42414, WO-A-02/32736, WO-A-02/28881 and WO-A-03/082807 (especially 2-aminomethyl-4-ethyl-hexanoic acid) and pharmaceutically acceptable salts and solvates thereof.

Other useful cyclic alpha-2-delta ligands for use in the present invention may be depicted by the following formula (I):

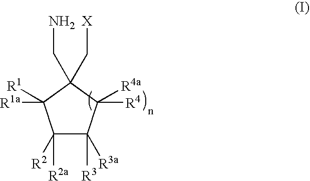

wherein X is a carboxylic acid or carboxylic acid bioisostere; n is 0, 1 or 2; and R¹, R^{1a}, R², R^{2a}, R³, R^{3a}, R⁴ and R^{4a} are independently selected from H and C₁-C₆ alkyl; or R¹ and R² or R² and R³ are taken together to form a C₃-C₇ cycloalkyl ring, which is optionally substituted with one or two substituents selected from C₁-C₆ alkyl; or a pharmaceutically acceptable salt or solvate thereof.

In formula (I), suitably, R¹, R^{1a}, R^{2a}, R^{3a}, R⁴ and R^{4a} are H and R² and R³ are independently selected from H and methyl, or R^{1a}, R^{2a}, R^{3a} and R^{4a} are H and R¹ and R² or R² and R³ are taken together to form a C₃-C₇ cycloalkyl ring, which is optionally substituted with one or two methyl substituents. A suitable carboxylic acid bioisostere is selected from tetrazolyl and oxadiazolonyl. X is preferably a carboxylic acid.

In formula (I), preferably, R¹, R^{1a}, R^{2a}, R^{3a}, R⁴ and R^{4a} are H and R² and R³ are independently selected from H and methyl, or R^{1a}, R^{2a}, R^{3a} and R^{4a} are H and R¹ and R² or R² and R³ are taken together to form a C₄-C₅ cycloalkyl ring, or, when n is 0, R¹, R^{1a}, R^{2a}, R^{3a}, R⁴ and R^{4a} are H and R² and R³ form a cyclopentyl ring, or, when n is 1, R¹, R^{1a}, R^{2a}, R^{3a}, R⁴ and R^{4a} are H and R² and R³ are both methyl or R¹, R^{1a}, R^{2a}, R^{3a}, R⁴ and R^{4a} are H and R² and R³ form a cyclobutyl ring, or, when n is 2, R¹, R^{1a}, R², R^{2a}, R³, R^{3a}, R⁴ and R^{4a} are H, or, n is 0, R¹, R^{1a}, R^{2a}, R^{3a}, R⁴ and R^{4a} are H and R² and R³form a cyclopentyl ring.

Further useful acyclic alpha-2-delta ligands for use in the present invention may be depicted by the following formula (II):

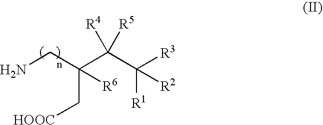

wherein n is 0 or 1, R¹ is hydrogen or (C₁-C₆)alkyl; R² is hydrogen or (C₁-C₆)alkyl; R³ is hydrogen or (C₁-C₆)alkyl; R⁴ is hydrogen or (C₁-C₆)alkyl, R⁵ is hydrogen or (C₁-C₆)alkyl and R² is hydrogen or (C₁-C₆)alkyl, or a pharmaceutically acceptable salt or solvate thereof.

According to formula (II), suitably R¹ is C₁-C₆ alkyl, R² is methyl, R³-R⁶ are hydrogen and n is 0 or 1. More suitably R¹ is methyl, ethyl, n-propyl or n-butyl, R² is methyl, R³- R⁶ are hydrogen and n is 0 or 1. When R² is methyl, R³- R⁶ are hydrogen and n is 0, R¹ is suitably ethyl, n-propyl or n-butyl. When R² is methyl, R³-R⁶ are hydrogen and n is 1, R¹ is suitably methyl or n-propyl. Compounds of formula (II) are suitably in the 3S,5R configuration.

Preferred alpha-2-delta ligands for use in the present invention include: gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline and (2S,4S)-4-(3-fluorobenzyl)proline and the pharmaceutically acceptable salts and solvates thereof. Pregabalin, or a pharmaceutically acceptable salt or solvate thereof is particularly preferred.

Further preferred alpha-2-delta ligands are (3R,4R,5R)-3-amino-4,5-dimethyl-heptanoic acid and (3R,4R,5R)-3-amino-4,5-dimethyl-octanoic acid and the pharmaceutically acceptable salts and solvates thereof. One of these compounds can be made using the following methods and the other compound can be made by analogous methods.

### (R)-3-((R)-3-Methyl-hexanoyl)-4-phenyl-oxazolidin-2-one

To a copper(I)bromide dimethylsulfide complex (13.34 g, 64.87 mmol) in dry tetrahydrofuran (150 ml) at −30° C. under nitrogen was added a 2M ether solution of propylmagnesiumchloride (64.87 ml, 129.7 mmol). The reaction mixture was stirred for 20 min. A solution of (R)-3-but-2-enoyl-4-phenyl-oxazolidin-2-one (15.0 g, 64.87 mmol) in tetrahydrofuran (60 ml) was added over a 15 minute period at −35° C. and the reaction mixture was allowed to slowly warm to room temperature over 4 hours. The mixture was cooled to 0° C. and quenched with saturated ammonium chloride solution. The resulting suspension was extracted into ether, washed with 5% ammonium hydroxide solution and brine and dried over MgSO₄. The solution was concentrated under reduced pressure to afford the title compound (13.34 g; 100%) as a white solid. ¹H NMR (400 MHz, CDCl₃) δ ppm 0.8 (m, 6 H) 1.2 (m, 3 H) 1.6 (s, 1 H) 2.0 (m, 1 H) 2.7 (dd, J=16.1, 8.5 Hz, 1 H) 3.0 (dd, J=15.9, 5.4 Hz, 1 H) 4.3 (dd, J=8.9, 3.8 Hz, 1 H) 4.7 (t, J=8.9 Hz, 1 H) 5.4 (dd, J=8.8, 3.9 Hz, 1 H) 5.4 (dd, J=8.8, 3.9 Hz, 1 H) 7.3 (m, 5 H). MS, m/z (relative intensity): 276 [M+1H, 100%].

### (R)-3-((2R,3R)-2,3-Dimethyl-hexanoyl)-4-phenyl-oxazolidin-2-one

To a 1M solution of sodium hexamethyldisylamide (16.2 g, 88.3 mmol) in tetrahydrofuran at −78° C. was added, via canular, a 0° C. solution of (R)-3-((R)-3-methyl-hexanoyl)-4-phenyl-oxazolidin-2-one (18.7 g 67.9 mmol) in 70 ml of dry tetrahydrofuran. The resulting solution was stirred at −78° C. for 30 min. Methyl Iodide (48.2 g, 339.5 mmol) was added and stirring at −78° C. was continued for 4 hours. The reaction mixture was quenched with saturated ammonium chloride solution, extracted into CH₂Cl₂ and washed with 1M sodium bisulfite. The solution was dried over MgSO4, concentrated and chromatographed in 10% ethylacetate in hexane to give the title compound (11.1 g, 56.5%) as an oil. ¹H NMR (400 MHz, CDCl₃) δ ppm 0.8 (t, J=7.0 Hz, 3 H) 0.9 (d, J=6.6 Hz, 3 H) 1.0 (d, J=6.8 Hz, 3 H) 1.0 (d, J=8.5 Hz, 1 H) 1.1 (m, 1 H) 1.4 (m, 1 H) 1.7 (m, 1 H) 3.7 (m, 1 H) 4.2 (dd, J=8.8, 3.4 Hz, 1 H) 4.6 (t, J=8.7 Hz, 1 H) 5.4 (dd, J=8.7, 3.3 Hz, 1 H) 7.2 (m, 2 H) 7.3 (m, 3 H). MS, m/z (relative intensity):290 [M+1H, 100%].

### (2R,3R)-2,3-Dimethyl-hexan-1-ol

A 1M solution of lithium aluminum hydride in tetrahydrofuran (95.9 ml, 95.9 mmol) was added to a solution of (R)-3-((2R,3R)-2,3-dimethyl-hexanoyl)-4-phenyl-oxazolidin-2-one in tetrahydrofuran (300 ml) under nitrogen at −78° C. The reaction mixture was stirred for 3 hours at that temperature. Water was added dropwise to quench the excess lithium aluminum hydride and the reaction mixture was then poured into a mixture of ice and ether. The resulting mixture was extracted into ether which was washed with water and dried over MgSO₄. The solution was concentrated followed by the addition of excess hexane. The resulting white precipitate was filtered and washed with hexane. The filtrate was concentrated to afford the title compound (5.05 g, 100%) as an oil. ¹H NMR (400 MHz, CDCl₃) δ ppm 0.9 (m, 9 H) 1.0 (d, J=6.8 Hz, 1 H) 1.1 (m, 1 H) 1.2 (m, 3 .4 (m, 1 H) 3.6 (m, 1 H).

### (2R,3R)-2,3-Dimethyl-hexanal

A mixture of pyridinium chlorochromate (27.35 g, 126.9 mmol) and neutral alumina (96 g, 3.5 g per gram of pyridinium chlorochromate) in dry dichloromethane (200 ml) was stirred under nitrogen for 0.25 hr. (2R,3R)-2,3-Dimethyl-hexan-1-ol (5.0 g, 38.46 mmol) in dichloromethane (60 ml) was added and the resulting dark slurry was stirred at room temperature for 3 hours. The slurry was filtered through a short pad of silica eluting with excess dichloromethane. Evaporation of the solvent afforded the title compound (4.1, 84%) as an oil. ¹H NMR (400 MHz, CDCl₃) δ ppm 0.8 (m, 3 H) 0.9 (d, J=6.6 Hz, 3 H) 1.0 (d, J=6.6 Hz, 3 H) 1.2 (m, 4 H) 1.8 (m, 1 H) 2.2 (m, 1 H) 9.6 (s, 1H).

### 4-Methyl-benzenesulfinic acid ((2R,3R)-2,3-dimethyl-hexylidene)-amide

Titanium(IV) ethoxide (5.16 g, 22.6 mmol) and (S)-(+)-p-toluenesulfinamide (7.02 g, 45.2 mmol) were added to (2R,3R)-2,3-dimethyl-hexanal (2.9 g, 22.6 mmol) in dry tetrahydrofuran (30 ml). The resulting mixture was stirred at room temperature for 18 hours and poured into a brine solution (40 ml). The resulting slurry was rapidly stirred for 10 minutes and filtered. The filtrate was extracted into ethyl acetate, and the extract was washed with brine and dried over MgSO₄. The solvent was evaporated and the residue was filtered through a silica plug, eluting with 50/50 solution of hexane/ethyl acetate to afford the title compound (3.1 g, 51.6%) as an oil. ¹H NMR (400 MHz, CDCl₃) δ ppm 0.8 (m, 6H) 1.1 (m, 4H) 1.3 (m, 3H) 1.7 (m, 1 H) 2.4 (s, 3 H) 2.5 (m, 1 H) 7.3 (d, J=8.3 Hz, 2 H) 7.5 (d, J=8.1 Hz, 2 H) 8.1 (d, J=5.4 Hz, 1 H). MS, m/z (relative intensity): 266 [M+1H, 100%].

### (4R,5R)-4,5-Dimethyl-(R)-3-(toluene-4-sulfinylamino)-octanoic acid tert-butyl ester

Butyl lithium (26.3 ml, 42.04 mmol) was added to a solution of diisopropylamine (4.6 g, 45.6 mmol) in dry tetrahydrofuran (40 ml) under nitrogen at 0° C. and the resulting mixture was stirred for 20 minutes. The solution was cooled to −78° C. followed by the addition of t-butyl acetate (4.1 g, 35.0 mmol) and stirred at that temperature for 45 minutes. Chlorotitanium triisopropoxide (9.4 g, 36.2 mmol) was added dropwise and stirring was continued for 30 minutes at −78° C. A −50° C. solution of 4-methyl-benzenesulfinic acid ((2R,3R)-2,3-dimethyl-hexylidene)-amide (3.1 g, 11.7 mmol) in dry tetrahydrofuran (10 ml) was added to the reaction and the resulting mixture was stirred at −78° C. for 4 hours. The e reaction was quenched with a saturated solution of NaH₂PO₄ and extracted into ethyl acetate. The extract was dried over MgSO₄ and concentrated. The resulting residue was chromatographed on silica, eluting with 15% ethyl acetate in hexane to give the title compound (2.4 g, 53.9%) as white solid. ¹H NMR (400 MHz, CDCl₃) δ ppm 0.9 (m, 6 H) 1.0 (d, J=6.6 Hz, 3 H) 1.1 (m, 1 H) 1.3 (m, 2 H) 1.4 ) m, 9 H) 1.5 (m, 2 H) 2.4 (s, 3 H) 2.6 (m, 2 H) 3.8 (m, 1 H) 4.4 (d, J=10.0 Hz, 1 H) 7.3 (d, J=8.1 Hz, 2 H) 7.6 (d, J=8.1 Hz, 2 H). MS, m/z (relative intensity): 382 [M+1H, 100%], 326 [M+1H-C(CH₃)₃, 50%].

### (3R,4R,5R)-3-Amino-4,5-dimethyl-octanoic acid

To a solution of (4R,5R)-4,5-dimethyl-(R)-3-(toluene-4-sulfinylamino)-octanoic acid tert-butyl ester (1.8 g, 4.71 mmol) in dry methanol (30 ml) at 0° C. under nitrogen was added excess trifluoroacetic acid (25 ml) and the reaction mixture was stirred for 2 hours at that temperature. The solution was concentrated to dryness followed by the addition of dry dichloromethane (20 ml) and trifluoroacetic acid (20 ml). The resulting mixture was stirred for 2 hours under nitrogen and concentrated to dryness. The residue was applied to BondElute SCX ion exchange resin and eluted with water until the eluent was at constant pH of 6.5. The resin was then eluted with a 1:1 solution of methanol and 10% ammonium hydroxide solution. The ammonium hydroxide solution was evaporated and the residue was crystallized with methanol-acetonitrile mixture to afford the title compound (0.717 g, 81.2%) as a white solid. ¹H NMR (400 MHz, CD₃OD) δ ppm 0.9 (m, 11 H) 1.1 (m, 2 H) 1.3 (m, 1 H) 1.4 (m, 1 H) 1.6 (m, 1 H) 1.7 (m, 2 H) 2.3 (dd, J=16.6, 10.0 Hz, 1 H) 2.5 (dd, J=16.7, 3.5 Hz, 1 H) 3.3 (m, 1 H). MS, m/z (relative intensity): 188 [M+1H, 100%], 186 [M-1 H, 100%].

In the context of this invention, a 5-HT_{1B}, 5-HT_{1D} or 5-HT_{1F} agonist is a compound which binds measurably to one or more of these three receptors and activates it to some extent (preferably binding with an affinity of less than 100 nM, most preferably less than 10 nM). Preferably, a 5-HT_{1B}, 5-HT_{1D} or 5-HT_{1F} agonist selected for use in the combination provided by the present invention is a selective 5-HT_{1B}, 5-HT_{1D} or 5-HT_{1F} agonist. A selective agonist may be defined as a compound that has a higher binding affinity (as measured by a K_{D} value) for one or more of the 5-HT_{1B}, 5-HT_{1D} and 5-HT1F receptors than for any 5-HT receptor other than the 5-HT_{1B}, 5-HT_{1D} and 5-HT_{1F} receptors. Selectivity over the 5-HT_{1A}, 5-HT_{2A}, 5-HT_{2C}, 5-HT₃, 5-HT₄, 5-HT_{5A} and 5-HT₆ receptors is preferred. The level of selectivity over these receptors is preferably at least 2 fold, more preferably at least 4 fold, more preferably still at least 10 fold and most preferably at least 100 fold. Binding affinity for one or more of the 5-HT receptors can be measured using the methods described in European Journal of Pharmacology, 1999, 368, 259 and Life Sciences, 1997, 61, 2117.

A particularly preferred 5-HT_{1B}, 5-HT_{1D} or 5-HT_{1F} agonist for use in the invention is a compound which is an agonist (preferably a selective agonist, as defined above) of both the 5-HT_{1B} receptor and the 5-HT_{1D} receptor (known as a 5-HT_{1B/1D} agonist). Such compounds include the indole-containing antimigraine drugs known as triptans, e.g. almotriptan, alnatriptan, avitriptan, donitriptan, frovatriptan, naratriptan, rizatriptan, sumatriptan and zolmitriptan and the pharmaceutically acceptable salts and solvates thereof.

The most preferred 5-HT_{1B}, 5-HT_{1D} or 5-HT_{1F} agonist for use in the invention is eletriptan and the pharmaceutically acceptable salts and solvates thereof, particularly eletriptan hydrobromide and eletriptan hemisulphate, most particularly the α-polymorphic form of eletriptan hydrobromide described in WO-A-96/06842 and the form I polymorph of eletriptan hemisulphate described in WO- A-01/23377.

Also preferred are selective agonists of the 5-HT_{1F} receptor (such as LY334370 ((5-(4-fluorobenzoyl)amino-3-(1-methylpiperidin-4-yl)-1H-indole fumarate) and LY344864). See Phebus et al, Life Sciences, 1997, 21, 2117 and Ramandan et al, Cephalalgia, 2003, 23, 776.

Other suitable 5-HT_{1B}, 5-HT_{1D} or 5-HT_{1F} agonists are PNU-109291 ((S)-(−)-1-[2-[4-(4-methoxyphenyl)-1-piperazinyl]ethyl]-N-methyl-isochroman-6-carboxamide), ergotamine, dihydroergotamine, IS-159, L-775606, L-772405, L-741604 and serotonin-O-carboxymethyl-glycyl-tyrosinamide.

In one embodiment, the invention provides a combination of a 5-HT_{1B} agonist (preferably a selective agonist, as defined above) and an alpha-2-delta ligand.

In another embodiment, the invention provides a combination of a 5-HT_{1D} agonist (preferably a selective agonist, as defined above) and an alpha-2-delta ligand.

In another embodiment, the invention provides a combination of a 5-HT_{1F} agonist (preferably a selective agonist, as defined above) and an alpha-2-delta ligand.

In another embodiment, the invention provides a combination of a 5-HT_{1B/1D} agonist (preferably a selective agonist, as defined above) and an alpha-2-delta ligand.

In another embodiment, the invention provides a combination of a 5-HT_{1B/1F} agonist (preferably a selective agonist, as defined above) and an alpha-2-delta ligand.

In another embodiment, the invention provides a combination of a 5-HT_{1D/1F} agonist (preferably a selective agonist, as defined above) and an alpha-2-delta ligand.

In another embodiment, the invention provides a combination of a 5-HT_{1B/1D/1F} agonist (preferably a selective agonist, as defined above) and an alpha-2-delta ligand.

A preferred combination according to the invention is a combination of a triptan antimigraine drug and an alpha-2-delta ligand.

Another preferred combination according to the invention is a combination of eletriptan, or a pharmaceutically acceptable salt or solvate thereof and an alpha-2-delta ligand.

Another preferred combination according to the invention is a combination of a 5-HT_{1B}, 5-HT_{1D} or 5-HT_{1F} agonist and an alpha-2-delta ligand selected from gabapentin, pregabalin, [(1R,5R,6S)-6-(aminomethyl)bicyclo[3.2.0]hept-6-yl]acetic acid, 3-(1-am inomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline, (2S,4S)-4-(3-fluorobenzyl)proline, (3R,4R,5R)-3-amino-4,5-dimethyl-heptanoic acid and (3R,4R,5R)-3-amino-4,5-dimethyl-octanoic acid and the pharmaceutically acceptable salts and solvates thereof.

Another preferred combination according to the invention is a combination of a 5-HT_{1B}, 5-HT_{1D} or 5-HT_{1F} agonist and pregabalin or a pharmaceutically acceptable salt or solvate thereof.

Another preferred combination according to the invention is a combination of a triptan antimigraine drug and an alpha-2-delta ligand selected from gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α) (3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline, (2S,4S)-4-(3-fluorobenzyl)proline, (3R,4R,5R)-3-amino-4,5-dimethyl-heptanoic acid and (3R,4R,5R)-3-amino-4,5-dimethyl-octanoic acid and the pharmaceutically acceptable salts and solvates thereof.

Another preferred combination according to the invention is a combination of a triptan antimigraine drug and pregabalin or a pharmaceutically acceptable salt or solvate thereof.

Another preferred combination according to the invention is a combination of 5-HT_{1B/1D} agonist (preferably a selective agonist, as defined above) and an alpha-2-delta ligand selected from gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-am inomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline, (2S,4S)-4-(3-fluorobenzyl)proline, (3R,4R,5R)-3-amino-4,5-dimethyl-heptanoic acid and (3R,4R,5R)-3-amino-4,5-dimethyl-octanoic acid and the pharmaceutically acceptable salts and solvates thereof.

Another preferred combination according to the invention is a combination of a 5-HT_{1B/1D} agonist and pregabalin or a pharmaceutically acceptable salt or solvate thereof.

A preferred specific combination according to the invention is the combination of eletriptan, or a pharmaceutically acceptable salt or solvate thereof, and gabapentin, or a pharmaceutically acceptable salt or solvate thereof.

A preferred specific combination according to the invention is the combination of eletriptan, or a pharmaceutically acceptable salt or solvate thereof, and pregabalin, or a pharmaceutically acceptable salt or solvate thereof.

A preferred specific combination according to the invention is the combination of eletriptan, or a pharmaceutically acceptable salt or solvate thereof, and [(1R,5R,6S)-6-(aminomethyl)bicyclo[3.2.0]hept-6-yl]acetic acid, or a pharmaceutically acceptable salt or solvate thereof.

A preferred specific combination according to the invention is the combination of eletriptan, or a pharmaceutically acceptable salt or solvate thereof, and 3-(1-aminomethyl-cyclohexylmethyl)-4H-[1,2,4]oxadiazol-5-one, or a pharmaceutically acceptable salt or solvate thereof.

A preferred specific combination according to the invention is the combination of eletriptan, or a pharmaceutically acceptable salt or solvate thereof, and C-[1-(1H-tetrazol-5-ylmethyl)-cycloheptyl]-methylamine, or a pharmaceutically acceptable salt or solvate thereof.

A preferred specific combination according to the invention is the combination of eletriptan, or a pharmaceutically acceptable salt or solvate thereof, and (3S,4S)-(1-aminomethyl-3,4-dimethyl-cyclopentyl)-acetic acid, or a pharmaceutically acceptable salt or solvate thereof.

A preferred specific combination according to the invention is the combination of eletriptan, or a pharmaceutically acceptable salt or solvate thereof, and (1α,3α,5α)(3-amino-methyl-bicyclo[3.2.0]hept-3-yl)-acetic acid, or a pharmaceutically acceptable salt or solvate thereof.

A preferred specific combination according to the invention is the combination of eletriptan, or a pharmaceutically acceptable salt or solvate thereof, and (3S,5R)-3-aminomethyl-5-methyl-octanoic acid, or a pharmaceutically acceptable salt or solvate thereof.

A preferred specific combination according to the invention is the combination of eletriptan, or a pharmaceutically acceptable salt or solvate thereof, and (3S,5R)-3-amino-5-methyl-heptanoic acid, or a pharmaceutically acceptable salt or solvate thereof.

A preferred specific combination according to the invention is the combination of eletriptan, or a pharmaceutically acceptable salt or solvate thereof, and (3S,5R)-3-amino-5-methyl-nonanoic acid, or a pharmaceutically acceptable salt or solvate thereof.

A preferred specific combination according to the invention is the combination of eletriptan, or a pharmaceutically acceptable salt or solvate thereof, and (3S,5R)-3-amino-5-methyl-octanoic acid, or a pharmaceutically acceptable salt or solvate thereof.

A preferred specific combination according to the invention is the combination of eletriptan, or a pharmaceutically acceptable salt or solvate thereof, and (2S,4S)-4-(3-chlorophenoxy)proline, or a pharmaceutically acceptable salt or solvate thereof.

A preferred specific combination according to the invention is the combination of eletriptan, or a pharmaceutically acceptable salt or solvate thereof, and 2-aminomethyl-4-ethyl-hexanoic acid, or a pharmaceutically acceptable salt or solvate thereof.

A preferred specific combination according to the invention is the combination of eletriptan, or a pharmaceutically acceptable salt or solvate thereof, and (2S,4S)-4-(3-fluorobenzyl)proline, or a pharmaceutically acceptable salt or solvate thereof.

A preferred specific combination according to the invention is the combination of eletriptan, or a pharmaceutically acceptable salt or solvate thereof, and (3R,4R,5R)-3-amino-4,5-dimethyl-heptanoic acid, or a pharmaceutically acceptable salt or solvate thereof.

A preferred specific combination according to the invention is the combination of eletriptan, or a pharmaceutically acceptable salt or solvate thereof, and (3R,4R,5R)-3-amino-4,5-dimethyl-octanoic acid, or a pharmaceutically acceptable salt or solvate thereof.

A 5-HT_{1B}, 5-HT_{1D} or 5-HT_{1F} agonist or an alpha-2-delta ligand selected for use in the combination of the present invention, particularly one of the suitable or preferred compounds listed above, (hereinafter referred to as ‘a compound for use in the invention’) may be used in the form of a pharmaceutically acceptable salt, for example an acid addition or base salt.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts of a compound for use in the invention may be prepared by one or more of three methods:

(i) by reacting the compound with the desired acid or base;

(ii) by removing an acid- or base-labile protecting group from a suitable precursor of the compound or by ring-opening a suitable cyclic precursor, for example, a lactone or lactam, using the desired acid or base; or

(iii) by converting one salt of the compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

A compound for use in the invention may exist in both unsolvated and solvated forms. The term ‘solvate’ is used herein to describe a molecular complex comprising the compound and a stoichiometric amount of one or more pharmaceutically acceptable solvent molecules, for example, ethanol. The term ‘hydrate’ is employed when said solvent is water.

A compound for use in the invention may form a complex such as a clathrate, a drug-host inclusion complexe wherein, in contrast to the aforementioned solvates, the drug and host are present in stoichiometric or non-stoichiometric amounts. A compound for use in the invention may also contain two or more organic and/or inorganic components which may be in stoichiometric or non-stoichiometric amounts. The resulting complexes may be ionised, partially ionised, or non-ionised. For a review of such complexes, see J. Pharm. Sci., 64 (8), 1269-1288, by Haleblian (August 1975).

A compound for use in the invention may be used in the form of a pro-drug. Thus, certain derivatives of a compound which may have little or no pharmacological activity themselves can, when administered into or onto the body, be converted into compounds having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association). Prodrugs can, for example, be produced by replacing appropriate functionalities with certain moieties known to those skilled in the art as ‘pro-moieties’ as described, for example, in Design of Prodrugs by H. Bundgaard (Elsevier, 1985).

A compound for use in the invention may also form active metabolites when administered to a patient, mainly by oxidative processes. Hydroxylation by liver enzymes is of particular note.

A compound for use in the invention which contains one or more asymmetric carbon atoms can exist as two or more stereoisomers. Where a compound contains an alkenyl or alkenylene group, geometric cisltrans (or Z/E) isomers are possible. Where structural isomers are interconvertible via a low energy barrier, tautomeric isomerism (‘tautomerism’) can occur. This can take the form of proton tautomerism in compounds containing, for example, an imino, keto, or oxime group, or so-called valence tautomerism in compounds which contain an aromatic moiety. It follows that a single compound may exhibit more than one type of isomerism.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, Stereochemistry of Organic Compounds by E. L. Eliel and S. H. Wilen (Wiley, New York, 1994).

compound for use in the invention may be isotopically-labelled wherein one or more atoms are replaced by atoms having the same atomic number, but an atomic mass or mass number different from the atomic mass or mass number which predominates in nature.

Examples of such isotopes include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³¹I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Pharmaceutically acceptable solvates include those wherein the solvent of crystallization may be isotopically substituted, e.g. D₂O, d₆-acetone, d₆-DMSO.

A compound for use in the invention may be administered as a crystalline or amorphous product. It may be obtained, for example, as a solid plug, powder or film by methods such as precipitation, crystallization, freeze drying, spray drying, or evaporative drying. Microwave or radio frequency drying may be used for this purpose.

A compound for use in the invention may be administered alone but will more likely be administered as a formulation in association with one or more pharmaceutically acceptable excipients. The term ‘excipient’ is used herein to describe any ingredient other than a compound for use in the invention. The choice of excipient will to a large extent depend on factors such as the particular mode of administration, the effect of the excipient on solubility and stability, and the nature of the dosage form.

Pharmaceutical compositions suitable for the delivery of a compound for use in the invention and methods for their preparation will be readily apparent to those skilled in the art. Such compositions and methods for their preparation may be found, for example, in Remington's Pharmaceutical Sciences, 19th Edition (Mack Publishing Company, 1995).

A compound for use in the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

A compound for use in the invention may also be used in a fast-dissolving, fast-disintegrating dosage form such as one of those described in Expert Opinion in Therapeutic Patents, 11 (6), 981-986, by Liang and Chen (2001).

For tablet dosage forms, depending on dose, a compound for use in the invention will generally make up from 1 weight % to 80 weight % of the dosage form, more typically from 5 weight % to 60 weight % of the dosage form. In addition, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound for use in the invention, a film-forming polymer, a binder, a solvent, a humectant, a plasticiser, a stabiliser or emulsifier, a viscosity-modifying agent and a solvent. Some components of the formulation may perform more than one function.

A compound for use in the invention may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, a compound for use in the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or release. delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

A compound for use in the invention may also be administered directly into the blood stream, into muscle, or into an internal organ. Such parenteral administration includes intravenous, intraarterial, intraperitoneal, intrathecal, intraventricular, intraurethral, intrasternal, intracranial, intramuscular and subcutaneous administration. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of a compound used in the preparation of parenteral solutions may be increased by the use of appropriate formulation techniques, such as the incorporation of solubility-enhancing agents. Formulations for parenteral administration may be formulated to be immediate and/or release. delayed-, sustained-, pulsed-, controlled-, targeted and programmed release. Thus a compound for use in the invention may be formulated as a solid, semi-solid, or thixotropic liquid for administration as an implanted depot providing modified release of the active compound. Examples of such formulations include drug-coated stents and poly(d/-lactic-coglycolic)acid (PGLA) microspheres.

A compound for use in the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci., 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or release. delayed-, sustained-, pulsed-, controlled-, targeted and programmed release.

A compound for use in the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of a compound for use in the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of a compound for use in the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound for use in the invention per actuation and the actuation volume may vary from 1 μl to 100 μl. A typical formulation may comprise a compound for use in the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations intended for inhaled/intranasal administration.

Formulations for inhaled/intranasal administration may be formulated to be immediate and/or release using, for example, PGLA delayed-, sustained-, pulsed-, controlled-, targeted and programmed release.

In the case of dry powder inhalers and aerosols, the dosage unit is determined by means of a valve which delivers a metered amount. Units are typically arranged to administer a metered dose or “puff”. The overall daily dose will be administered in a single dose or, more usually, as divided doses throughout the day.

A compound for use in the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or release. delayed-, sustained-, pulsed-, controlled-, targeted and programmed release.

A compound for use in the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or release delayed-, sustained-, pulsed-, controlled-, targeted, and programmed release.

A compound for use in the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO-A-91/11172, WO-A-94/02518 and WO-A-98/55148.

The two components of the present combination invention (i.e. the 5-HT_{1B}, 5-HT_{1D} or 5-HT_{1F} agonist and the alpha-2-delta ligand) may be administered simultaneously, sequentially or separately in order to enjoy the benefits of the combination therapy provided by the present invention. Each component may be administered on its own but is more usually administered in association with one or more excipients as one of the pharmaceutical compositions described above. Usually, both components will be administered via the same route (e.g. the oral route). However, there may be circumstances where it is preferable to administer each component via a different route (e.g. one component via the oral route and one component via the parenteral route). For simultaneous administration, the two components preferably form part of the same pharmaceutical composition and are therefore administered via the same route.

Oral administration is preferred for both components of the invention. Most preferably, the two components are delivered simultaneously via the oral route, for example in the form of a tablet

The two components of the present combination invention may conveniently be combined in the form of a kit. Such a kit comprises a 5-HT_{1B}, 5-HT_{1D} or 5-HT_{1F} agonist and an alpha-2-delta ligand, each usually in the form of one of the pharmaceutical compositions described above, and means for separately retaining them, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering separate compositions at different dosage intervals, or for titrating separate compositions against one another. To assist compliance, the kit typically comprises directions for administration and may be provided with a so-called memory aid.

For administration to human patients, the optimal total daily dose of the 5-HT_{1B}, 5-HT_{1D} or 5-HT_{1F} agonist and the alpha-2-delta ligand administered according to the present invention will vary considerably according to the particular compounds chosen. Such optimal doses are readily determined by the skilled person in accordance with normal pharmaceutical practice using dose ranging studies. As an example, in the case where the chosen 5-HT_{1B}, 5-HT_{1D} or 5-HT_{1F} agonist is eletriptan, the total daily oral dose is typically in the range 20 mg to 80 mg. The administration of one or two 40 mg doses is particularly preferred. In the case where the alpha-2-delta ligand is pregabalin, the total daily oral dose is usually from 150 to 600 mg, taken as two or three doses.

The total daily dose of either component may be administered in single or divided doses and may, at the physician's discretion, fall outside of the typical ranges described above.

For the avoidance of doubt, references herein to “treatment” include references to curative, palliative and prophylactic treatment.

Some of the advantages of the combination provided by the present invention may be appreciated in a pre-clinical models (especially preclinical models of migraine pathophysiology or central sensitisation).

Such models include:
the rat model for cutaneous allodynia induced by intracranial pain described by Burstein et al in Annals of Neurology, 2004, 55(1), 27-36;
the animal model of intracranial pain described by Ramadan in Proceedings of the National Academy of Sciences of the United States of America, 2003, 101(12), 4274-9;
the rat model described by Burstein et a/ in Journal of Neurophysiology, 1999, 81(2), 479-93; and
the rat model described by Burstein et a/ in Journal of Neurophysiology, 1998, 79(2), 964-82.

The-advantages of the combination provided by the present invention will also be apparent from clinical measurements of efficacy. In the case of migraine headache such advantages can be seen as improved efficacy (e.g. the rate of migraine resolution) and as an improved safety profile (e.g. in the reduction in the adverse events).

A combination of eletriptan and pregabalin has been tested in the rat model of migraine developed by Burnstein and disclosed in the Journal of Neurophysiology references cited above. This sensitization model uses chemical mediators of inflammation applied to the dura to induce a headache in the rat. The chemical mediators (serotonin, 10⁻³M; histamine, 10⁻³M; prostaglandin E2, 10⁻⁴M and bradykinin 10⁻³M) are applied in a combined preparation referred to as an inflammatory soup. The progress of the headache is monitored using electrophysiology of a 2^{nd} sensory neuron in the trigeminal nucleus caudalis (TNC). In this model, once sensitization is induced, it is not reversed by the actions of triptans (including eletriptan). This model therefore reflects the clinical observation that after allodynic symptoms have developed during a migraine attack, the triptans often do not relieve all of the patient's pain.

A control animal was treated with the inflammatory soup on the dura at time 0 and then with saline solution 3 hours later. This animal showed strong sensitization of its responses to sensory stimuli such as brush and pin. The receptive fields increased and there was a large increase in the number of action potentials at 2.5 hours after sensitization was induced. At 4.5 hours after the application of the inflammatory soup, the sensitization was stable and an increase in the magnitude of the response to the sensory stimuli was maintained.

Animals treated with inflammatory soup on the dura followed by eletriptan at 3 hours showed strong sensitization of their responses at 2.5 hours to sensory stimuli such as brush and pin and the eletriptan did not reverse the sensitization even as late as 5.5 hours after sensitization. This is in accordance with clinical studies of the effects of triptans on sensitization and allodynia in patients, which have shown that in the approximately 80% of patients who experience allodynia during their migraine, the triptans are much less effective if treatment is delayed until after sensitization is manifest.

Animals treated with inflammatory soup on the dura followed by pregabalin (30 mg/kg) at 3 hours showed sensitization of their responses at 2.5 hours to sensory stimuli such as brush and pin and the pregabalin moderately reversed the sensitization at the 4.5 hour time points after sensitization. This change was not consistent among the animals tested. There was a large amount of variability between the animals and they did not show a smooth return to baseline activity at the 3.5 hour and the 4.5 hour time points.

However, in animals treated with inflammatory soup on the dura followed by a combination of pregabalin (30 mg/kg) and eletriptan (0.2 mg/kg) at 3 hours, the combination of drugs reversed sensitization and the number of spikes in response to the same sensory stimulus at 4.5 hours after sensitization was less then before application of the soup.

The data show, in a rat model of migraine, that although triptans alone do not reverse sensitization of trigeminal relay neurons in the TNC, a combination of the triptan eletriptan and the alpha-2-delta ligand pregabalin is effective.

A combination of the present invention may be further combined with another pharmacologically active compound, or with two or more other pharmacologically active compounds, particularly in the treatment of pain, especially migraine. Thus, a combination of the present invention, in its broadest sense or in any of the preferred aspects presented above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, naetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl[-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local an aesthetic such as mexiletine;
a corticosteroid such as dexamethasone;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4- hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3- amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine;
a 5-HT3 antagonist, such as ondansetron;
   and the pharmaceutically acceptable salts and solvates thereof.

1. A combination of a 5-HT1B, 5-HT1D or 5-HT1F agonist and an alpha-2-delta ligand.

2. A combination as claimed in claim 1 wherein the 5-HT1B, 5-HT1D or 5-HT1F agonist is a 5-HT1B/1D agonist.

3. A combination as claimed in claim 2 wherein the 5-HT1B/1D agonist is a triptan antimigraine drug.

4. A combination as claimed in claim 3 wherein the triptan antimigraine drug is eletriptan, or a pharmaceutically acceptable salt or solvate thereof.

5. A combination as claimed in claim 1 wherein the alpha-2-delta ligand is pregabalin, or a pharmaceutically acceptable salt or solvate thereof.

6. A combination as claimed in claims 1 for use as a medicament.

7. A pharmaceutical composition comprising a combination as defined in claim 1 and a pharmaceutically acceptable excipient, diluent or carrier.

8. (canceled)

9. A method of treating pain comprising administering simultaneously, sequentially or separately, to a mammal in need of such treatment, an effective amount of a 5-HT1B, 5-HT1D or 5-HT1F agonist and an alpha-2-delta ligand.

10. The method of claim 9 wherein the pain is migraine pain.

11. The method of claim 9 wherein the 5-HT1B, 5-HT1D or 5-HT1F agonist is eletriptan or a pharmaceutically acceptable salt or solvate thereof.

12. The method of claim 9 wherein the alpha-2-delta ligand is pregabalin or a pharmaceutically acceptable salt or solvate thereof.

13. A kit comprising a 5-HT1B, 5-HT1D or 5-HT1F receptor agonist, an alpha-2-delta ligand and means for containing said compounds.
